(19) United States
(12) Patent Application Publication
(10) Pub. No.: US 2005/0048065 A1
(43) Pub. Date: Mar. 3, 2005

(54) Methods and compositions containing antigens having a targeting moiety specific for antigen presenting cells for intranasal immunization

(76) Inventor: Denis P. Snider, Hamilton (CA)

Correspondence Address: SIM & MCBURNEY, 330 UNIVERSITY AVENUE, 6TH FLOOR, TORONTO, ON, M5G 1R7, CA

(21) Appl. No.: 10/937,403

(22) Filed: Sep. 10, 2004

Related U.S. Application Data
(63) Continuation of application No. 08/634,039, filed on Apr. 17, 1996, now abandoned.

(51) Int. Cl. A61K 39/395

(52) U.S. Cl. 424/178.1

(57) ABSTRACT

An immune response to an antigen in a host is elicited by intranasal administration to the host an antigen coupled to a targeting moiety generally a monoclonal antibody or fragment therof, specific for surface structures of antigen-presenting cells, such as class I or class II MHC cells, B-cells, T-cells, dendritic cells and CD4⁺ cells.

### FIELD OF THE INVENTION

The present invention is related to the field of immunology and is particularly concerned with the intranasal administration of antigens having a targeting moiety specific for antigen presenting cells to evoke an immune response.

### BACKGROUND OF THE INVENTION

Current theories of immunology suggest that, in order to provide a potent antibody response, an antigen must be seen by both B cells, which subsequently develop into the antibody producing cells, and also by helper T-cells, which provide growth and differentiation signals to the antigen specific B-cells. Helper T-cells recognize the antigen on the surface of antigen-presenting cells (APC) in association with Class II major histocompatibility complex (MHC) gene products.

There are significant advantages in using proteins and peptides and other antigens such as polysaccharides derived from proteins of infectious organisms as components in subunit vaccines. The search for such suitable subunits constitutes a very active area of both present and past research. Advances in techniques of recombinant DNA manipulations, antigen and protein purification, peptide synthesis and cellular immunology have greatly assisted in this endeavour. However, a problem in the use of such materials as vaccines has been the relatively poor in-vivo immunogenicity of most protein subunits, polysaccharides and peptides. Generally, the immune response to vaccine preparations is enhanced by the use of adjuvants. However, the only currently licensed adjuvants for use in humans are aluminum hydroxide and aluminum phosphate, collectively termed alum, which is limited in its effectiveness as a potent adjuvant. There is thus a need for new adjuvants with the desired efficacy and safety profiles.

Several adjuvants, such as Freund's Complete Adjuvant (FCA), syntex and QS21, have been used in animals (ref 1—Throughout this application, various references are referred to in parenthesis to more fully describe the state of the art to which this invention pertains. Full bibliographic information for each citation is found at the end of the specification, immediately preceding the claims. The disclosures of these references are hereby incorporated by reference into the present disclosure). A novel way of engaging both the B and T cell components of an immune response has been described, which uses anti-class II monoclonal antibodies (mabs) coupled to antigens to target class II bearing antigen presenting cells (APC's) (refs 2 to 4, also U.S. Pat. Nos. 4,950,480 and 5,194,254). Experiments carried out in-vivo in rodents and rabbits using this technology, (refs. 2 to 5), have demonstrated convincing proof of enhancement in immunogenicity of antigens, in the absence of conventional adjuvants. Other cell surface markers such as Surface Immunoglobulin (sIg) (ref. 6), and MHC class I (refs. 7, 8), have been used to achieve targeting to APC's.

Production of local secretory IgA and systemic IgG is thought to be important in the prevention or reduction of morbidity and mortality during viral or bacterial respiratory infections (ref. 9). Efficacious vaccines must elicit an immune response to protect the host by neutralizing and eliminating intruders quickly, or by priming the immune response to respond rapidly during subsequent infections. Indeed, the presence of these Ig isotypes is often the best hallmark of immunity at mucosal epithelia (ref. 10).

Intranasal (IN) inoculation of antigen (Ag) has been explored as a means to immunize the nasopharyngeal mucosa and lungs (refs. 11 to 14). Both local IgA and circulating IgG has been produced after IN exposure to model protein (ref. 15, 16), bacterial (refs. 14, 17 to 19) or viral antigens (refs. 20 to 22). IN administration of soluble protein Ag alone usually does not elicit substantial antibody or cellular immune responses (refs. 15, 16, 23 to 25). These failures may be overcome to some extent by co-administration of Ag with adjuvants, such as cholera toxin (CT) or its B subunit (refs. 13, 22, 26, 27), or by formulation of Ag in liposomes (ref. 28), ISCOMS (ref. 29) or microparticles (ref. 19). If subunit vaccines are to be effective when administered by the IN route, enhancing the immunogenicity of protein Ag is desirable to provide efficacious immunity in the respiratory tract.

It would be advantageous to provide methods and compositions containing antigens having a targeting moiety specific for antigen presenting cells for intranasal immunization for generating immune responses including protective immune responses, and in diagnostic applications.

### SUMMARY OF THE INVENTION

The present invention enables an immune response to an antigen to be generated in a host by coupling the antigen to a targeting moiety specific for surface structures of antigen-presenting cells and intranasally administering the resulting immunogenic molecule. It is surprising that a strong immune response to the antigen can be evoked by intranasal administration and the success in eliciting a good immune response to the antigen by parenteral adminstration to the antigen as described in the U.S. Pat. Nos. 4,950,480 and 5,194,254 referred to above is not in any way predictive of the results obtained herein.

Accordingly, in one aspect of the present invention, there is provided a method of generating an immune response to an antigen in a host, which comprises intranasally administering to the host an antigen coupled to a targeting moiety specific for surface structures of antigen-presenting cells. Such antigen-presenting cells may be selected from the group consisting of class I or class II major histocompatibility expressing cells (MHC), B-cells, T-cells or professional antigen-presenting cells including dendritic cells and CD4⁺ cells.

The targeting moiety generally comprises a monoclonal antibody or a fragment thereof. The antigen may be coupled to the monoclonal antibody by physical coupling as specifically-described in the Barber patents (U.S. Pat. No. 4,950,480 and U.S. Pat. No. 5,194,254) or by the use of heterobifunctional cross-linking agents as described in more detail below. Alternatively, the antigen and monoclonal antibody may be coupled by recombinant means which genetically modify the antibody moiety to contain the antigen, as described more particularity in co-pending U.S. patent application Ser. No. 08/483,576, filed Jun. 7, 1995, assigned to the assignee hereof and the disclosure of which is incorporated herein by reference.

The antigen which is administered in accordance with the present invention may comprise any protein, peptide, carbohydrate or ligand or any portion or fragment thereof against which an immune response may be evoked by coupling to the targeting moiety and administering the molecule intranasally.

In one preferred embodiment of the invention, the antigen is derived from a pathogen and the immune response is a protective immune response, which may be an IgG and/or an IgA immune response, against the pathogen, in a host, including primates and humans.

The immunogenic composition may be administered in any convenient form. In accordance with another aspect of the present invention, there is provided, in combination with a disperser for dispersing as an aerosol, atomized spray or liquid drops for intranasal administration to generate an immune response in a host, a composition comprising an immunologically-effective amount of an immunogenic molecule comprising an antigen coupled to a targeting moiety specific for surface structures of antigen-presenting cells and a pharmaceutically-acceptable carrier suitable for intranasal administration.

In one aspect the present invention provides the use of an immunogen comprising an antigen and a targeting moiety specific for surface structures of antigen-presenting cells for the manufacture of a medicament for intranasal administration to a host to generate an antigen-specific immune response.

One feature of the present invention is the ability to obtain a strong immune response to an antigen by intranasal administration.

### BRIEF DESCRIPTION OF THE DRAWINGS

The present invention will be further understood from the following general description and specific Examples with reference to the Figures in which:

FIG. 1 shows an electrophoretic analysis of monoclonal antibody-hen egg lysozyme (HEL) conjugates;

FIG. 2, comprising panels (a), (b), (c) and (d), shows serum antibody responses in mice immunized with monoclonal antibody HEL conjugates;

FIG. 3, comprising panels (a) and (b), shows a comparison of serum antibody responses in mice inoculated with monoclonal antibody-HEL conjugates or with cholera toxin adjuvant;

FIG. 4, comprising panels (a), (b) and (c), shows the anti-HEL IgA responses in mice immunized with monoclonal antibody-HEL conjugates;

FIG. 5, comprising panels (a), (b) and (c), shows the secondary antibody responses by mice primed with monoclonal antibody-HEL conjugates followed by 1N HEL challenge in the presence or absence of cholera toxin (CT).

### GENERAL DESCRIPTION OF THE INVENTION

The principal determinant of specific immunity at mucosal surfaces is secretory IgA (S-IgA) which is physiologically and functionally separate from the components of the circulatory immune system. S-IgA antibody responses may be induced locally by the application of suitable immunogens to a particular mucosal site. The bulk of mucosal S-IgA responses, however, are the results of immunity generated via the common mucosal immune system (CMIS) (ref. 30), in which immunogens are taken up by specialized lympho-epithelial structures, collectively referred to as mucosa-associated lymphoid tissue (MALT). The best studied immunologic lympho-epithelial structures are the gut-associated lymphoid tissues (GALT), such as intestinal Peyer's patches. It is now clear, however, that other structurally and functionally similar lymphoid follicles occur at other mucosal surfaces, including those of the respiratory tract (ref. 31).

Bronchus-associated lymphoid tissue (BALT) was described by Bienenstock (refs. 32, 33) in experimental animals, but is apparently not present in the noninfected human bronchial tree (ref. 34). The upper respiratory tract in humans, however, is furnished with Waldeyer's ring of tonsils and adenoids. In rodents, the functional equivalent of these consists of nasal-associated lymphoid tissue (NALT), a bilateral strip of lymphoid tissue with overlying M cell-like epithelial cells at the base of the nasal passages (ref. 35).

In the present invention, an antigen, against which it is desired to raise an immune response including antibodies in a host, is coupled to a targeting moiety specific for surface structures of antigen presenting cells. The targeting molecule may be a monoclonal antibody. The monoclonal antibody, therefore, acts as a “vector” or “delivery vehicle” for targeting antigenic determinants to antigen presenting cells, thereby facilitating their recognition by T-helper cells. Antigen presenting cells possess a variety of specific cell surface structures or markers which are targeted by any particular targeting moiety such as a monoclonal antibody or fragment thereof. Thus, antigens may be coupled to a monoclonal antibody specific for any of the surface structures on the antigen presenting cells that internalize antigen into the cells, including class I and class II major histocompatibility complex (MHC) gene products. Other antigen-presenting cells include dendritic cells and CD4⁺ cells.

The surface structures on the antigen presenting cells of the immune system which can be recognized and targeted are numerous and the specific surface antigen structure targeted depends on the specific targeting moiety including monoclonal antibodies and fragments thereof.

The monoclonal antibody may be specific for a gene product of the MHC, and, in particular, may be specific for class I molecules of MHC or for class II molecules of MHC. However, the invention is not limited to such specific surface structures and the conjugates containing the corresponding monoclonal antibodies, but rather, as will be apparent to those skilled in the art, the invention is applicable to any other convenient surface structure of antigen presenting cells which can be recognized and targeted by a specific antibody or fragment thereof to which an antigenic molecule is coupled and which internalize antigen into the cells.

For example, strong adjuvant-independent immune responses to a delivered antigen can be obtained with conjugates formed with a dendritic cell-specific monoclonal antibody and a CD4⁺ cell-specific monoclonal antibody.

In the present invention, the monoclonal antibody specific for the target structure is provided in the form of a conjugate with an antigen against which it is desired to elicit an immune response. Such antigen may be joined to the C-terminus of the heavy and/or light chains of the monoclonal antibody or to free lysine residues by covalent linkage. While the conjugate antibody molecules are illustrated by such C-terminal connection, the antigen moiety alternatively may be inserted within the light and heavy chains of the antibody and such insertions may establish a particular constrained conformation of the antigen and, in particular, epitopes, within the known structural framework of an antibody molecule. Such conjugate antibody molecules may be conveniently produced by genetic modification of a gene encoding the heavy and light chains of the antibody to contain a gene encoding one or more antigen(s) and coexpressing the resulting nucleic acid molecules, as described in the aforementioned U.S. application Ser. No. 08/483,576, or by conjugation, as described in the aforementioned Barber patents (U.S. Pat. No. 4,950,480 and U.S. Pat. No. 5,194,254), or by covalent linkage by coupling to free lysine using heterobifunctional cross linking reagents, as described herein.

The invention is particularly useful for antigen molecules which normally possess a weakly-immunogenic response, since the response is potentiated by the present invention. The antigen molecule may be in the form of a peptide, protein or carbohydrate, but is not limited to such materials.

The present invention is applicable to any antigen which it is desired to target to antigen presenting cells using the monoclonal antibody or fragment thereof or other targeting moiety. The antigen may be a protein or a peptide of 6 to 100 amino acids comprising an amino acid sequence of an epitope. Representative organisms from which the antigen may be derived include viruses such as influenza viruses, parainfluenza viruses, respiratory viruses, measles viruses, mumps viruses, human immunodeficiency viruses, polio viruses, rubella viruses, herpex simplex viruses type 1 and 2, hepatitis viruses types A, B and C, yellow fever viruses, smallpox viruses, rabies viruses, vaccinia viruses, reo viruses, rhinoviruses, Coxsackie viruses, Echoviruses, rotaviruses, papilloma viruses, paravoviruses and adenoviruses; bacteria such as E. coli, V. cholera, BCG, M. tuberculosis, C. diphtheria, Y. pestis, S. typhi, B. pertussis, S. aureus, S. pneumoniae, S. pyogenes, S. mutans, Myocoplasmas, Yeasts, C. tetani, meningococci (e.g., N. meningitidis), Plasmodium spp, Mycobacteria spp, Shigella spp, Campylobacter spp, Proteus spp, Neisseria gonorrhoea, and Haemophilus influenzae; and other microorganisms such as Mycoplasmas, yeasts and plasmodium species. The antigen moiety may also be derived from hormones, such as human HCG hormone, and tumor-associated antigens. Polysaccharide antigens including (LOS) and polyribosylphosphate (PRP) may also be employed.

The immunogenic molecule comprising the antigen coupled to a targeting moiety specific for surface structures of antigen-presenting cells may be formulated for intranasal administration as an immunogenic composition with a pharmacologically acceptable carrier, such as water, buffered saline, ethanol, polyol, for example, glycerol, propylene glycol or liquid polyethylene glycol, suitable mixtures thereof, or vegetable oils. If necessary, various antibacterial and antifungal agents, such as parabens, chlorobutanol, phenol, sorbic acid and thimerosal may also be used as preservatives. It may be also preferable to include in the formulation isotonic agents, for example, glucose or sodium chloride. Such formulation may be administered intranasally as an aerosol or atomized spray, or as liquid drops.

As used herein, “pharmacologically acceptable carrier” includes any and all solvents, dispersion media, antibacterial and antifungal agents, isotonic and absorption delaying agents which may be appropriate for intranasal administration of the immunogenic molecules. The use of such media and agents for pharmaceutically active substances is known in the art. Except insofar as any conventional media or agent is incompatible with the active ingredient, its use in the compositions administered intranasally is contemplated.

It is especially advantageous to formulate the immunogenic composition in dosage unit form for ease of administration and uniformity of dosage. “Dosage unit form” as used herein refers to a physically discrete unit of immunogenic composition appropriate for the subject to be immunized. Each dosage should contain the quantity of active material calculated to produce the desired therapeutic effect in association with the selected pharmacologically-acceptable carrier. Procedures for determining the appropriate vaccine dosage for a given class of recipient are well known to those skilled in the art. Generally, when administering the immunogenic composition, a dosage of about 1-500 μg of antigen should be satisfactory for producing the desired immune response.

The experimental data presented herein and detailed in the Examples below show clearly that the immunotargeting approach to antigen delivery is very effective. As little as 0.1 μg of hen egg lyzozyme (HEL), given twice as a component of an immunotargeting conjugate with anti-MHC-II (anti-IA^{k}) IgG2b monoclonal antibodies, was sufficient to prime mice for a secondary humoral immune response to HEL. By comparison, very little antibody response was seen in mice immunized with up to 10 μg of HEL alone. In addition, at an equivalent HEL dose, the targeting conjugate primed mice for greater secondary antibody responses than HEL in the presence of CT, one of the strongest mucosal adjuvants known (refs. 36, 37). The dose response results indicated that the secretory IgA response was more sensitive than serum IgG response to priming by the targeting conjugate. Thus, while both serum IgG and IgA declined with decreasing dose, specific antibodies in nasal washings stayed at a relatively high level even with the lowest dose used. The applicants experimental results provided herein demonstrate that immunotargeting is a very potent means of stimulating IgA memory for local secretory responses in the respiratory tract.

The intranasal administration of an immunogen comprising an antigen and a targeting moiety to generate an antigen-specific immune response of the present invention is useful for the generation of antigen-specific antibodies for use in immunoassays including enzyme-linked immunosorbent assays (ELISA), RIAs and other non-enzyme linked antibody binding assays or procedures known in the art for the detection of antigen. In ELISA assays, the antigen-specific antibodies are immobilized onto a selected surface, for example, a surface capable of binding proteins such as the wells of a polystyrene microtiter plate. After washing to remove incompletely adsorbed antigen-specific antibodies, a nonspecific protein that is known to be antigenically neutral with regard to the test sample, such as a solution of bovine serum albumin (BSA), may be bound to the selected surface. This allows for blocking of nonspecific adsorption sites on the immobilizing surface and thus reduces the background caused by nonspecific bindings of antisera onto the surface.

The immobilizing surface is then contacted with a sample, such as clinical or biological materials, to be tested in a manner conducive to immune complex (antigen/antibody) formation. This may include diluting the sample with diluents, such as solutions of BSA, bovine gamma globulin (BGG) and/or phosphate buffered saline (PBS)/Tween. The sample is then allowed to incubate for from about 2 to 4 hours, at temperatures such as of the order of about 20° to 37° C. Following incubation, the sample-contacted surface is washed to remove non-immunocomplexed material. The washing procedure may include washing with a solution, such as PBS/Tween or a borate buffer. Following formation of specific immunocomplexes between the test sample and the bound antigen-specific antibodies and subsequent washing, the occurrence, and even amount, of immunocomplex formation may be determined by subjecting the immunocomplex to a second antibody having specificity for the antigen. To provide detecting means, the second antibody may have an associated activity such as an enzymatic activity that will generate, for example, a colour development upon incubating with an appropriate chromogenic substrate. Quantification may then be achieved by measuring the degree of colour generation using, for example, a spectrophotometer.

### EXAMPLES

The above disclosure generally describes the present invention. A more complete understanding can be obtained by reference to the following specific Examples. These Examples are described solely for purposes of illustration and are not intended to limit the scope of the invention. Changes in form and substitution of equivalents are contemplated as circumstances may suggest or render expedient. Although specific terms have been employed herein, such terms are intended in a descriptive sense and not for purposes of limitations.

### Example 1

This Example describes the preparation and analysis of antigen-antibody conjugates.

Hen egg lysozyme (HEL) was obtained from Sigma Chemical Co. (St. Louis, USA). The hybridoma 10-2.16, secreting anti-MHC II (anti-IA^{k}) IgG2b monoclonal antibodies (mAbs) was obtained from the American Type Culture Collection (ATCC; Rockville, USA). These antibodies were purified from ascites fluids by ammonium sulphate precipitation and high performance liquid chromatography as described in ref. 38. Purified IgG2b control mAb, designated PV-144 and having irrelevant Ag specificity was kindly provided by Dr. Ursula McGuiness, Connaught Laboratories Limited (Toronto, Canada).

The HEL-mAb conjugates were prepared as described previously (refs. 38, 39) using the heterobifunctional crosslinking reagent N-succinimidyl 3-(2-pyridydithio)proprionate (Pierce; Rockville, USA). The HEL-mAb conjugates were separated from free HEL using a Sephadex G-75 (Pharmacia, Uppsala, Sweden) column equilibrated in tris-hydroxymethylaminomethane (TRIS)-buffered saline (TBS; 0.02 M Tris, 0.15 M NaCl, pH 7.2). Following dialysis into sterile, phosphate-buffered saline (PBS) (0.01 M NaPO₄, 0.15 M NaCl, pH 7.4) and concentration using Centricon-100 concentrators (Amicon, Oakville, Canada), the conjugates were assessed quantitatively for endotoxin contamination using the Cell Culture™ test kit (Sigma). Contaminating endotoxin was removed by incubation with Polymyxin B-coated agarose beads (Sigma). All stock conjugates had less than 1 ng/ml of endotoxin.

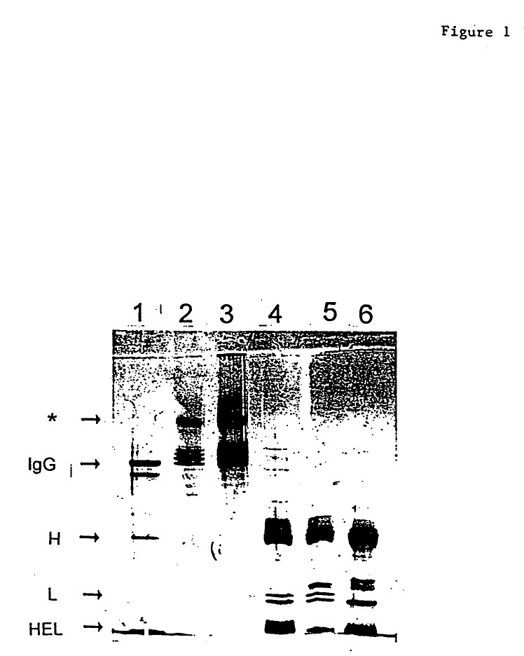

Isolated HEL-mAb conjugates were analyzed by 1-D sodium dodecylsulphate (SDS) polyacrylamide gel electrophoresis (PAGE) using the Phastsystem™ (Pharmacia). Samples were diluted in sample buffer (0.2 M Tris, 2% (w/v) SDS, pH 8.3). Reducing buffer consisted of sample buffer containing 0.5% (w/v) dithiothrietol (Sigma). All samples were incubated at 100° C. for 5 min. After cooling, reduced samples were alkylated with 1% (w/v) iodoacetamide (Sigma) and all samples stored in darkness for 1 h. at room temperature. Aliquots of 1 μL were applied to precast 4-15% (w/v) PAGE gels (Pharmacia) and electrophoresed using SDS-buffer strips (0.2 M Tricine, 0.2 M Tris, 0.55% (w/v) SDS, pH 8.1 in 2.8% (w/v) agarose). Following electrophoresis, the gels were silver-stained (ref. 40) to reveal polypeptides. The results of this analysis are shown in FIG. 1 in which. Lanes 1-3 and 4-6 show the results from non-reduced and reduced samples, respectively. Samples were separated on a 4-15% gradient gel in SDS and silver-stained. Lanes 1 and 4 contained a non-conjugated mixture of anti-MHC class II (I-A^{K}) IgG2b and HEL. Lanes 2 and 5, anti-MHC class II (I-A^{K}) IgG2b-HEL conjugate. Lanes 3 and 5, irrelevant IgG2b-HEL conjugate. H, heavy chain of IgG; L, light chain of IgG; * indicates high MW aggregates. Under non-reducing electrophoretic conditions free IgG2b and HEL were clearly separable (FIG. 1, lane 1). However, following conjugation and purification, free HEL was not detected. Rather, a defined series of increasing molecular weight bands was present, beginning coincidentally with free IgG. This indicated that from 1 to 4 HEL molecules were conjugated to each mAb molecule (lanes 2 and 3). This interpretation was strengthened by the observation that separation of free IgG H and L chains plus HEL in the conjugates occurred under reducing conditions that eliminated all bands of HEL-mAb conjugates (lanes 4 to 6). Under non-reducing conditions, the presence of high molecular weight material (asterisk) indicated that there were some large aggregates of HEL with more than one mAb molecule. HEL in the conjugates was readily demonstrable by enzyme immunoassay. These results demonstrate that HEL-mAbs conjugates were successfully prepared.

### Example 2

This Example describes the immunization of mice with the antigen-antibody conjugates.

Female A/J mice, aged 5 to 7 wks, were purchased from the Jackson Laboratory (Bar Harbor, USA) and used at ages 6 to 8 wks. Mice were inoculated IN with conjugates, HEL alone, HEL mixed with 1 μg of cholera toxin (CT) (List Biological Laboratories, Campbell, Calif.) or sterile PBS. Animals were restrained and 5 μL applied to each nare. The mice aspirated the inocula upon release from restraint. For priming with conjugates or control samples, inoculations were given on days 0 and 7. In some experiments, mice were inoculated subcutaneously with 50 μL in each hind flank. Mice were challenged IN using HEL with or without CT on day 25. Doses of immunogens varied with each experiment and were adjusted for HEL content as determined by enzyme immunoassay.

Sera were prepared from blood obtained via the retro-orbital plexus on days 21, 35 and day 40. Nasal and lung washings were prepared using a solution of PBS at 4° C. containing 0.05 TIU/ml Aprotinin (Sigma), 2 mM phenylmethylsulfonylfluoride, 5 mM ethylenediaminetetracetic acid (EDTA) and 0.02% (w/v) NaN₃. Mice were euthanized by anaesthetic overdose and the chests opened from the sternums to the necks. The tracheas were ligated with a 3-0 silk suture, and PE-50 polyethylene tubing inserted into the nasopharyngeal cavities. Contents of the nasal passages were then washed out of the nares with 0.5 mL of sampling solution. Lung washing was accomplished by inserting a 27 gauge needle into the trachea and the lungs were flushed 2-3 times with sampling solution, to a total volume of 1.0 ml per animal.

The conjugates were assayed for HEL content using a solid-phase “sandwich”-type ELISA requiring two mAbs specific for separate HEL epitopes (ref. 41). Other ELISA assays measured anti-HEL IgG1, IgG2a, IgG2b, or IgA titres in the sera, lung or nasal washings (ref. 40). Briefly, microtitre plate wells were coated with 10 μg/ml of HEL and post-coated with 1% (w/v) bovine serum albumin (BSA) (Sigma). The wells were washed with Tris-Tween buffer (TBS containing 0.05% (w/v) Tween-20). A 3-fold dilution series of each sample was prepared using TBS-BSA (TBS containing 0.1% (w/v) BSA, pH 7.2) as diluent and incubated overnight at 4° C. To measure IgA, sera dilutions began at 1:20 and washings at 1:10. To measure IgG subclasses, sera dilutions began at 1:50 and washings at 1:10. Following washing, biotinylated, affinity-purified-goat anti-mouse IgG subclass or IgA antibodies (Southern Biotechnology, Birmingham, USA) were added to the wells and incubated at 37° C. for 1 h. When tested against a panel of myeloma proteins, the anti-IgG subclass and IgA reagents showed less than 1% cross-reactivity and each exhibited a sensitivity of 1 to 3 ng/ml in detecting the appropriate myeloma isotype bound to microtitre plates. The wells were washed prior to addition of streptavidin-conjugated alkaline phosphatase and p-nitrophenyl phosphate substrate (Sigma) was added. The optical density (O.D.) of each well at 405 nm was determined using a Titertek Multiskan Plus (ICN Labsystems, Finland). Antibody titres were defined as the greatest dilution which produced a value at least 2-fold greater than the mean value of wells processed without sera or washings.

The statistical significance of differences in antibody titres were determined by comparing logarithmic-transformed geometric titres in a 2-tailed Student's test (p≦0.05). For samples without a detectable signal, values representing the minimum dilution titres were used.

### Example 3

This Example describes the serum antibody responses to immunization with conjugates.

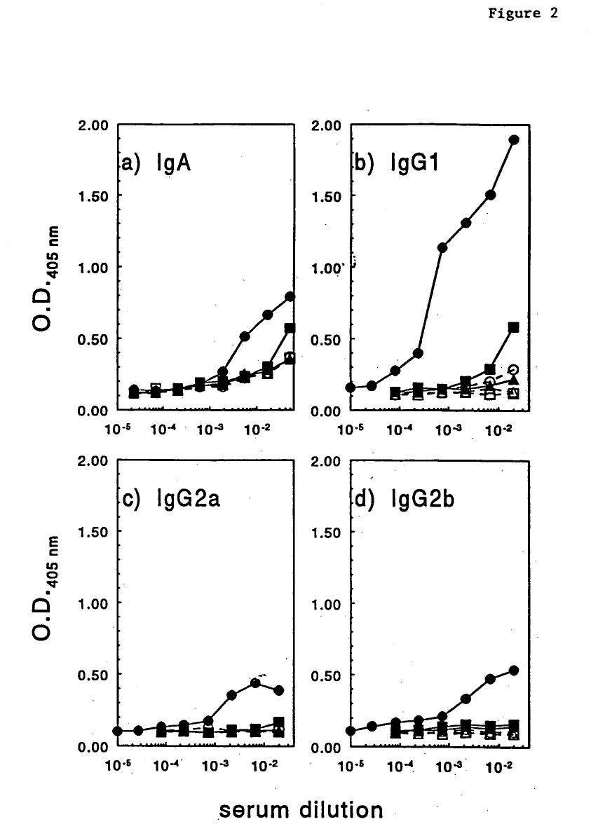

Female A/J mice were immunized IN with either the HEL-anti-MHC Class II IgG2b conjugate or the control IgG2b conjugate on days 0 and 7. Each inoculum contained the same amount of HEL as determined by enzyme immunoassay. Mice were immunized IN with HEL plus CT on day 25 and sera were obtained on days 21 and 35, pooled and analyzed for anti-HEL antibodies. The results are shown in FIG. 2. A/J mice were inoculated intranasally (IN) on days 0 and 7 with HEL-anti-MHC class II (I-AK) IgG2b conjugate (circles), irrelevant IgG2b-HEL conjugate (squares), or phosphate-buffered saline (triangles). Mice were challenged IN with 5 μg of HEL plus 1.0 μg of cholera toxin on day 25. Pooled sera was obtained on days 21 (open symbols) and 35 (closed symbols) and diluted as shown (x-axis). Mean O.D. values (y-axis) for duplicate samples are shown. The results show that appreciable amounts of specific IgA and very large amounts of specific IgG1 were detected in the sera of mice given HEL-anti-MHC II conjugates. This result from pooled samples was verified by comparison of titres from individual sera (Table 1). Little, if any, IgG1, IgG2a and IgA were detected in sera prior to challenge (FIG. 2), thus indicating very limited initial response to the conjugates. Small, yet significant amounts of IgG2a and IgG2b were detected only after challenge (FIG. 2). Mice receiving the control IgG2b conjugate had no detectable primary response, but had a small amount of specific IgA and IgG1 after challenge (Table 1). Those titres were approximately 50-fold less than those found with the targeting conjugate. Results shown in Table 1 confirmed by 3 similar experiments. Thus, a much greater priming of response occurred when using the non-targeting conjugate than when using the non-targeting conjugate. Sera taken on days 21 and 35 from control mice that received PBS as a primary inoculation, and given HEL and CT had no detectable IgA or IgG1 antibody (Table 2). These results indicated that mice given the HEL-anti-MHC II conjugate produced a substantial secondary antibody response, and were not simply responding to the HEL plus CT adjuvant challenge.

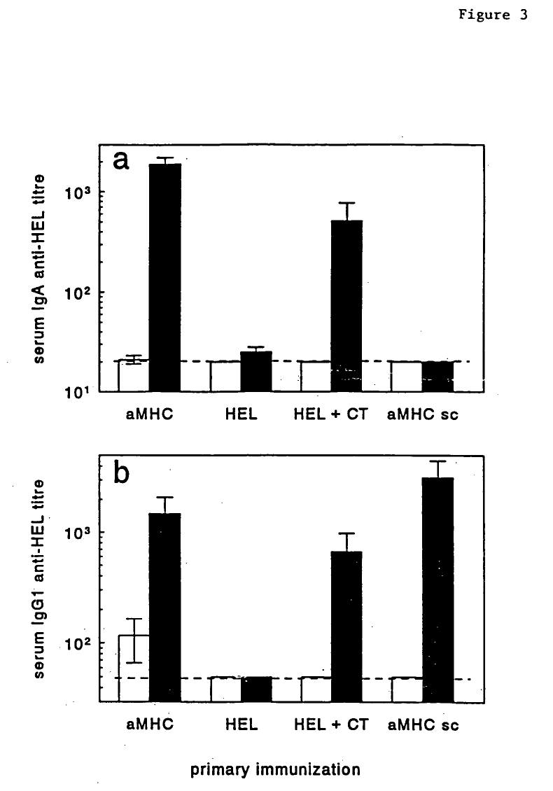

Additional control immunizations were conducted in separate experiments. A/J mice inoculated on days 0 and 7 with up to 10 μg of HEL alone failed to produce any anti-HEL antibodies in sera. Primary immunization of A/J mice with HEL alone did not lead to detectable anti-HEL IgG1 or IgA in sera after challenge with HEL plus CT (FIG. 3 and Table 2). In FIG. 3, A/J mice were immunized intranasally (IN) on days 0 and 7 with HEL-anti-MHC class 11 (I-A^{K}) IgG2b conjugate (anti-MHC), HEL alone, or HEL plus cholera toxin (HEL+CT). 2.5 μg of HEL was present in each inoculum. A separate group of mice was immunized twice subcutaneously (S.C.) with the anti-MHC class II conjugate containing 1.0 μg of HEL. IN immunized mice were given 10 μg of HEL plus 1.0 μg of CT IN on day 25 with subcutaneously immunized mice were given 10 μg of HEL S.C. Sera obtained on days 21 (open bars) and 35 (solid bars) were analyzed for anti-HEL antibodies by enzyme immunoassay. Data represent arithmetic mean±S.E.M. Comparison of the anti-MHC groups to HEL or HEL+CT on day 35 revealed significantly (p<0.03) more HEL-specific antibodies in sera obtained from the anti-MHC groups. As positive controls, some A/J mice were immunized twice with 10 μg of HEL plus CT and given HEL plus CT on day 25. These animals had strong secondary IgG1 and IgA anti-HEL serum responses (FIG. 3). However, those immunized with the HEL-anti-MHC II conjugates had even larger responses, thus indicating the anti-MHC class II targeting was as efficacious as CT in priming for a humoral immune response (FIG. 3 and Table 2). Finally, some mice were immunized subcutaneously (S.C.) with the targeting conjugate. When given HEL plus CT subcutaneously, strong IgG1 serum responses resulted, but no IgA responses were detectable. Thus, the delivery of the targeting conjugate IN provoked priming for serum IgA responses, but failed to do so when given S.C. The serum antibody response primed by IN immunotargeting was principally the IgG1 subclass, an isotype that is consistent with a Th2 cell cytokine response dominated by IL-4 and possibly including other Th2-type cytokines.

It is uncertain as to whether the dominance of IgG1 is important to immunization in the murine respiratory tract (ref. 42). In viral infection, IgG responses containing high affinity, neutralizing specificities would be beneficial. However, if viral or bacterial opsonization is required, then IgG2a or IgG2b would be needed (refs. 43, 44). We observed that some of these latter subclasses were produced after priming with HEL anti-MHC II conjugates.

The MHC-class II molecule specificity of the conjugate determined the targeting efficiency. The antibody titres following immunization with the anti-MHC class II conjugate were considerably higher than those induced by the non-targeting conjugate. Since we used a control mAb of the same isotype as the anti-MHC class II mAb, and the resulting conjugate showed nearly identical electrophoretic characteristics, neither isotype nor preparative differences define the disparate immunogenicity of the two conjugates. Use of the identical isotype also allowed that both conjugates interacted equally with any FcR expressed by immune cells involved in the targeting mechanism, such as macrophages or B cells.

### Example 4

This Example describes the local secretory IgA in nasal and lung washings to immunization with conjugates.

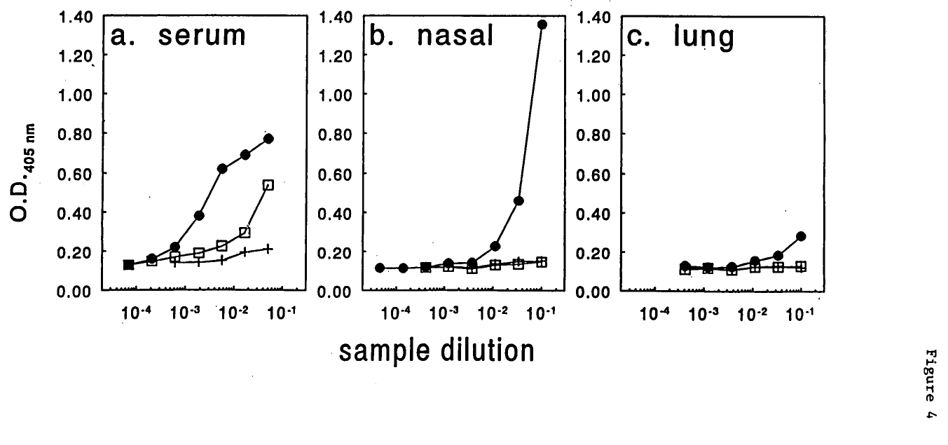

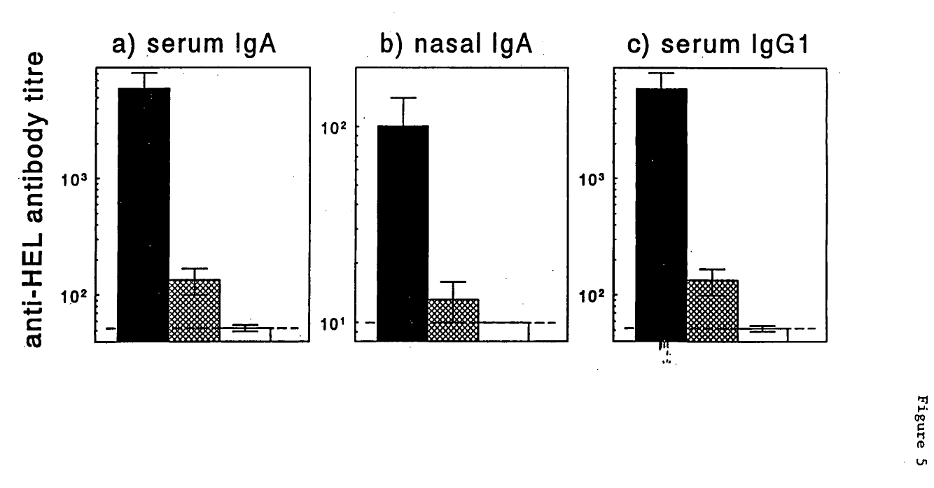

An examination of pooled samples from nasal and lung washings indicated that appreciable amounts of anti-HEL IgA were present in mice primed with the targeting conjugate, but not with the non-targeting conjugate (FIG. 4). In FIG. 4, A/J mice were immunized intranasally (IN) on days 0 and 7 with HEL anti-MHC class II (1-A^{K}) IgG2b conjugate (solid circles), with HEL conjugated to irrelevant monoclonal IgG2b (open squares) or with phosphate buffered saline (crosses). The animals were challenged on day 25 with HEL plus cholera toxin. Sera, nasal and lung washing pools were obtained on day 35 and diluted as shown (x-axis). Mean O.D. values (y-axis) for duplicate dilutions of each sample are shown. To confirm that the nasal anti-HEL IgA was secreted locally in the respiratory tract, specific IgG1 in the nasal and lung washings were quantitated. Anti-HEL IgG1 was not detected in nasal or lung washings, indicating that the specific IgA in such fluids was locally produced and was not a serum transudate. Indeed, if anti-HEL IgA in the washings was derived from serum, high titres of IgG1 would be expected also given the amounts of these two sera isotypes (FIG. 3, Table 1). Previous studies showed CT to be a strong adjuvant for IN immunization (refs. 12, 45), CT was included in these studies. However, if the anti-MHC-class II targeting is to be truly effective, it may have to prime for mucosal antibody responses in a manner that allows a strong secondary response to Ag alone in the absence of any adjuvant. Thus, we examined whether mice primed with the anti-MHC class II targeting conjugate would respond to a challenge with HEL alone. FIG. 5 shows that challenge with HEL alone could elicit a smaller but significant secondary response in respect of serum IgA and IgG1 when compared to a typical challenge with HEL plus CT. However, nasal washings did not show nearly as striking an IgA response when HEL was given alone. In FIG. 5, A/J mice were inoculated IN on days 0 and 7, the targeting conjugate, and challenged on day 25 with 10 μg HEL plus CT (solid bars), with 60 μg HEL alone (hatched bars), or with PBS (open bars). Sera and nasal washings were obtained from individual mice on day 35, and anti-HEL antibody titres determined by immunoassay. Data represent arithmetic mean±S.E.M. A significant difference was noted in all cases when comparing HEL plus CT to HEL alone (p<0.01).

Mice immunized with HEL anti-MHC II conjugates received 2.5 or 5 μg of HEL. This dose was similar to the oral dose used previously (ref. 38), yet the antibody response to IN inoculation was much more pronounced. In order to determine how efficient the targeting conjugates were via the IN route, mice were immunized with doses of conjugate containing as little as 0.1 μg of HEL. As shown in Table 2, doses as low as 0.5 μg and 0.1 μg were able to prime for detectable anti-HEL IgA in nasal washings and for anti-HEL IgA, IgG1, and IgG2a in sera. These doses represent extremely low amounts of Ag to the murine immune system. Immunotargeting by the IN route was capable of priming for a secondary antibody response, if protein Ag was administered by itself in the challenge, albeit lower than that noted in the presence of the CT adjuvant. This is a significant result because it suggests that a secondary antibody sequence can occur upon antigen exposure, but that additional adjuvant or inflammatory process could enhance that response. Therefore, a challenge with infectious agent may trigger a rapid secondary antibody response once sufficient viral or bacterial Ag is detected by memory B and T cells within the local tissues, especially when inflammatory events typical of infection were initiated.

The pathway by which the anti-MHC class II conjugates reach in the nasal immune system is not known. The lymphoid aggregates or NALT in rat nasal passages contain follicular-like structures and resemble to some extent the Peyer's patches of the intestine (refs. 66, 67). Some histologic evidence suggests that epithelial cells resembling intestinal M cells are part of the epithelium overlying the NALT. (ref. 45). Intestinal M cells are known for selective uptake of particulate Ag and for expression of immunoglobulin receptors (refs. 48 to 50). The targeting conjugates may gain specific access to the lymphoid aggregates through such cells. Alternatively, conjugates may be transported through or between the normal epithelial cells as there is some evidence that the nasal epithelium, in contrast to other sites, is relatively permeable to proteins, with reduced occurrence or function of tight junctions.

### SUMMARY OF THE DISCLOSURE

In summary of this disclosure, the present invention provides a method and means for intranasal immunization of a host, including humans, by coupling an antigen to a targeting moiety, particularly a monoclonal antibody, specific for surface structures of antigen-presenting cells. Modifications are possible within the scope of this invention.

TABLE 1
Antibody Responses in Sera and Nasal Washings After Immunization of
Mice with HEL-anti-MHC class II (I-A^{K}) IgG2b Conjugates

| | | Primary Inoculant^{a} | | |
|---|---|---|---|---|
| | | HEL anti-MHC-II | Irrelevant | |
| Specimen | Isotype | IgG2b Conjugate | IgG2b Conjugate | PBS |
| Serum | anti-HEL | 680 ± 240 (6/6)^{b} | 63 ± 27 (3/6) | <20 (0/6) |
| | IgA | | | |
| Nasal | anti-HEL | 89 ± 34 (6/6) | 18 ± 9 (1/6) | <10 (0/6) |
| Washing | IgA | | | |
| Serum | anti-HEL | 5280 ± 2070 | 77 ± 17 (3/6) | <50 (0/6) |
| | IgG1 | (6/6)^{c} | | |
| Nasal | anti-HEL | <10 (0/6) | 10 (0/6) | <10 (0/6) |
| Washing | IgG1 | | | |
| ^{a}Animals received intranasal (IN) doses of conjugate containing 5 μg of hen egg lysozyme (HEL) on days 0 and 7. Challenge was conducted IN on day 25 using 10 μg of | | | | |
| # free HEL plus 1.0 μg of cholera toxin. Sera and nasal washings were collected on day 35 and analyzed by enzyme immunoassay. | | | | |

^{b}Data are arithmetic mean titre (±S.D.). Quotient of responding mice shown in parentheses. Underlined values indicate significant differences (p < 0.01) compared to PBS-inoculated group.
^{c}Indicates significant differences (p < 0.03) compared to irrelevant IgG2b conjugate-inoculated groups.

TABLE 2
Efficient Priming of Anti-Hen Egg Lysozyme Antibody Responses Using
Low Immunizing Doses of HEL-Anti-MHC Class II (I-A^{K})
IgG2b Conjugates

| Priming | | | | IgA Response |
|---|---|---|---|---|
| Dosage | Serum Antibody Response^{b} | | | in Nasal |
| Form^{a} | IgG1 | IgG2a | IgA | Washings |
| HE-MHC class | | | | |
| II IgG2b | | | | |
| Conjugate | | | | |
| Containing: | | | | |
| 2.5 μg HEL | 1480 ± 600^{c} | 500 ± 190 | 1047 ± 390 | 240 ± 106 |
| | (6/6) | (6/6) | (6/6) | (5/5) |
| 0.5 μg HEL | 1770 ± 380 | 520 ± 280 | 851 ± 330 | 250 ± 69 |
| | (5/5) | (4/5) | (5/5) | (5/5) |
| 0.1 μg HEL | 302 ± 120 | 140 ± 55 | 125 ± 45 | 270 ± 30 |
| | (5/6) | (5/6) | (5/6) | (6/6) |
| 10 μg HEL | <50− | 57 ± 9 | 46 ± 39 | 22 ± 5 |
| (alone) | (0/5) | (2/5) | (2/5) | (2/5) |
| 10 μg HEL | 643 ± 301 | 300 ± 210 | 240 ± 140 | 87 ± 50 |
| plus CT | (4/4) | (3/4) | (3/4) | (3/4) |
| ^{a}Intranasal (IN) priming inoculations performed on days 0 and 7. | | | | |
| ^{b}IN challenge inoculations performed using 10 μg of HEL plus 1.0 μg cholera toxin (CT) on day 25. Sera and lung washings were collected on day 40 and analyzed by | | | | |
| # enzyme immunoassay. Data are arithmetic mean titer (±S.D.). | | | | |

^{c}Indirect significant differences for the HEL alone group (p < 0.03). Quotient of responding mice shown in parentheses.

### REFERENCES

(1) Warren, H. S.; Vogel, F. R. and Chedid, L. A. A.; Ann. Rev. Immun. (1986) 4:369-388.
(2) Carayanniotis, G. and Barber, B. H.; Nature (Lond.) (1987) 327:59-61.
(3) Carayanniotis, G.; Vizi, E.; Parker, J. M. R.; Hodger, R. S. and Barber, B. H.; Mol. Immunol. (1988) 25:907-911.
(4) Skea, D. L.; Douglas, A. R.; Skehel, J. J. and Barber, B. H.; Vaccine (1993) 11:994-999.
(5) Carayanniotis, G.; Skea, D. L.; Luscher, M. A. and Barber, B. H.; Mol. Immunol. (1991) 28:261-267.
(6) Kawamura, H. and Berzofsky, J. A.; J. Immunol. (1986) 136:58-65.
(7) Casten, L. A.; Kawnaya, P. and Pierce, S. K.; J. Exp. Med. (1988) 168:171-180.
(8) Casten, L. A. and Pierce, S. K.; J. Immunol. (1988) 140:404-410.

(9) Kilian, M. and Russell, M. W.; Handbook of Mucosal Immunology (Eds. Ogra, P. L.; Mesteck, J.; Lamm, M. E.; Strober, W.; McGhee, J. R.; and Bienenstock, J.;) Academic Press, San Diego, (1994) 127-140.
(10) McDermott, M. R.; Befus, A. D, and Bienenstock, J.; Int. Rev. Exp. Pathol. (1982) 23:47-112.
(11) Tamura, S.; Kurata, H.; Funato, H.; Nagamine, T.; Aizawa, C. and Kurata, T.; Vaccine (1989) 7:314-320.
(12) Nedrud, J. G.; Liang, X.; Hague, N. and Lamm, M. E.; J. Immunol. (1987) 139:3484-3492.
(13) Wu, H. Y. and Russell, M. W.; Infect. Immun. (1993) 61:314-322.
(14) Bessen, D. and Fischetti, V. A.; J. Immunol. (1990) 145:1251-1256.
(15) Hameleers, D. M. H.; van der Ven, I.; Biewenga, J. and Sminia, T.; Immunol. Cell Biol. (1991) 69:119-125.
(16) Aramaki, Y.; Fujii, Y.; Kikuchi, H. and Tsuchiya, S.; Vaccine (1994) 12:1241-1245.
(17) Wu, H. -Y. and Russell, M. W.; Vaccine (1994) 12:215-222.
(18) Langermann, S.; Palaszynski, S.; Sadziene, A.; Stover, C. K. and Koenig, S.; Nature (1994) 372:552-555.
(19) Cahill, E. S.; O'Hagan, D. T.; Illum, L.; Barnard, A.; Mills, K. H. G. and Redhead, K.; Vaccine (1995) 13:455-462.
(20) Tamura, S. I.; Samegai, Y.; Kurata, H.; Kikuta, K.; Nagamine, T.; Aizawa, C. and Kurata, T.; Vaccine (1989) 7:257-262.
(21) Reuman, P. D.; Keely, S. P. and Schiff, G. M.; J. Med. Virol. (1991) 35:192-197.
(22) Tamura, S.; Yamanaka, A.; Shimohara, M.; Tomita, T.; Komase, K.; Tsuda, Y.; Suzuki, Y.; Nagamine, T.; Kawahara, K.; Danbara, H.; Aizawa, C.; Oya, A. et al; Vaccine (1994) 12:419-426.
(23) Hirabayashi, Y.; Kurata, H.; Funato, H.; Nagamine, T.; Aizawa, C.; Tamura, S.; Shimada, K. and Kurata, T.; Vaccine (1990) 8:243-248.
(24) Loevgren, K., Kaberg, H. and Morein, B. Clin Exp. Immunol. (1990) 82:435-439
(25) Oka, T.; Honda, T.; Morokuma, K.; Ginnaga, A.; Ohkuma, K. and Sakoh, M.; Vaccine (1994) 12:1255-1258.
(26) Lipscombe, M.; Charles, I. G.; Roberts, M.; Dougan, G.; Tite, J. and Fairweather, N. F.; Mol. Microbiol. (1991) 5:1385-1392.
(27) Hajishengallis, G.; Hollingshead, S. K.; Koga, T. and Russell, M. W.; J. Immunol. (1995) 154:4322-4332.
(28) De Haan, A.; Geerligs, H. J.; Huchshorn, J. P.; Van Scharrenburg, G. J. M.; Palache, A. M. and Wilschut, J.; Vaccine (1995) 13:155-182.
(29) van Heyningen, S.; Science (1974) 183:656-657.
(30) Mestecky, J.; J. Clin. Immunol. (1987) 7:265-276.
(31) Croitoru, K.; Bienenstock. J.; Handbook of Mucosal Immunology, San Diego, Calif.: Academic Press, Inc. (1994) 141-149.
(32) Bienenstock, J.; Johnston, N.; Perey, D. Y.; Lab. Invest. (1973) 28:686-692.
(33) Bienenstock, J.; Johnston, N.; Perey, D. Y.; Lab. Invest. (1973) 28:693-698.
(34) Pabst R.; Immunology Today (1992) 13:119-122.
(35) Kuper, C. F., Koornstra, P. J.; Hameleers, D. M. H.; Biewenga, J.; Spit, B. J.; Duijvestijn, A. M.; van Breda Vriesman, P. J. C., and Sminia, T. Immunol. Today (1992) 13:219-224.
(36) Elson, C. O. and Ealding, W.; J. Immunol. (1984) 132:2736-2742.
(37) Lycke, N. and Holmgren, J. Immunology (1986) 59:301-307.
(38) Estrada, A.; McDermott, M. R.; Underdown, B. J. and Snider, D. P.; Vaccine (1995) 13:901-907.
(39) Mjaaland, S. and Fossum, S.; Int. Immunol. (1991) 3:1315-1321.
(40) Heukeshoven, J. and Dernick, R.; Electrophoresis (1988) 28-32.
(41) Snider, D. P.; Marshall, J. S.; Perde, M. H. and Liang, H.; J. Immunol. (1994) 153: 647-657.
(42) Graham, M. B.; Dalton, D. K.; Giltinan, D.; Braciale, V. L.; Steward, T. A. and Braciale, T. J.; J. Exp. Med. (1993) 178:1725-1732.
(43) Fazekas, G.; Rosenwirth, B.; Dukor, P.; Gergely, J.; and Rjnavolgyi, E.; Eur. J. Immunol. (1994) 24:3063-3067.
(44) Iwasaki, T. and Nozima, T.; J. of Immunol. (1997) 118:256-263.
(45) Fujihashi, K.; Yamamoto, M.; McGhee, J. R. and Kiyono, H.; J. Immunol. (1993) 151:6681-6691.
(46) Kuper, C. F.; Koornstra, P. J. Hameleers, D. M. H.; Biewenga, J.; Spit, B. J.; Duijvestijn, A. M.; Van Breda Vriesman, P. J. C. and Sminia, T.; Immunol. Today (1992) 13:219-224.
(47) Spit, B. J.; Hendriksen, E. G. J.; Bruijntjes, J. P. and Kuper, C. F.; Cell Tissue Res. (1989) 255:193-198.
(48) Owen, R. L.; J. Exp. Med. (1994) 180:7-9.
(49) Neutra, M. R. and Kraehenbuhl, J. -P.; Trends Cell Biol. (1992) 2:134-137.
(50) Weltzin, R.; Lucia-Jandris, P.; Michetti, P.; Fields, B. N.; Kraehenbuhl, J. P. and Neutra, M. R.; J. Cell Biol. (1989) 108:1673-1685.

1. A method of generating an immune response to an antigen in a host, which comprises:
intranasally administering to said host an antigen coupled to a targeting moiety specific for surface structures of antigen-presenting cells.

2. The method of claim 1 wherein said antigen-presenting cells are selected from the group consisting. of class I or class II major histocompatibility expressing cells (MHC), B-cells, T-cells, professional antigen-presenting cells including dendritic cells, and CD4⁺ cells.

3. The method of claim 2 wherein the targeting moiety is a monoclonal antibody or a fragment thereof.

4. The method of claim 3 wherein the antigen is a protein, peptide, carbohydrate or ligand.

5. The method of claim 4 wherein the antigen is derived from a pathogen and said immune response is a protective immune response against disease caused by said pathogen.

6. The method of claim 5 wherein the immune response is an IgG or an IgA immune response.

7. The method of claim 5 wherein the host is a human host.

8. The method of claim 1 wherein said antigen is coupled to said targeting moiety through a heterobifunctional linking molecule.

9. In combination with a disperser for dispersing as an aerosol, atomized spray or liquid drops for intranasal administration to generate an immune response in a host, a composition comprising an immunologically-effective amount of an immunogenic molecule comprising an antigen coupled to a targeting moiety specific for surface structures of antigen-presenting cells and a pharmacologically-acceptable carrier suitable for intranasal administration.
